(19) United States
(12) Patent Application Publication
(10) Pub. No.: US 2011/0245586 A1
(43) Pub. Date: Oct. 6, 2011

(54) Wellness System

(76) Inventor: Rodney Dale Slane, Stuart, FL (US)

(21) Appl. No.: 12/750,439

(22) Filed: Mar. 30, 2010

(51) Int. Cl. A61M 21/00

(52) U.S. Cl. 600/28

(57) ABSTRACT

A personal wellness system for supporting a person and subjecting the person to frequencies that are understood by sound therapy practitioners as being associated with synchronization with the mind and body. The wellness system comprises a support for receiving a person comprising a head portion, a back portion, a seat portion, and a foot portion. The wellness system further comprises plural transducers distributed in or on the support. Each transducer is capable of receiving a discrete control signal and outputting a single frequency vibration at an independent amplitude. The wellness system also comprises one or more speakers for outputting music or single frequency sounds. Additionally, the wellness system comprises a menu-driven control system that sends a signal to the transducers and coordinates speaker output with transducer output.

### TECHNICAL FIELD

The present invention relates to devices and methods for wellness, and more particularly to a wellness system used for sound therapy.

### BACKGROUND

Vibrating massage chairs often provide a choice of vibration frequency. Vibration of massage chairs may be performed by a variety of oscillating or rotating mechanisms, such as induction or eccentric means. Other seats, such as for use with video games, may also include vibrating means.

Sound chairs use low frequency transducer technology to create vibration. These types of transducers differ from the oscillating or rotating mechanisms used in vibrating massage and gaming chairs. The types of transducers used in sound chairs also differ from typical speakers used in home stereo equipment. Instead, these transducers emit low frequency vibrations that resonate with the human body through sound waves.

Typical sound chairs use combined frequencies from transducers with limited frequency response. The present invention instead uses pure frequencies with full frequency response. In this way, the present invention avoids splitting sound and allows for manipulation of specific frequencies to interact with certain regions of the body.

### SUMMARY

This Summary is provided to introduce a selection of concepts in a simplified form that are further described below in the Detailed Description of Illustrative Embodiments. This Summary is not intended to identify key features or essential features of this invention, nor is it intended to be used to limit the scope of the invention.

Additional features and advantages will be made apparent from the following detailed description of illustrative embodiments that proceeds with reference to the accompanying drawings.

The present invention is a personal wellness system for supporting a person and subjecting the person to frequencies that are understood by sound therapy practitioners as being associated with synchronization with the human body. The system comprises a support for receiving a person. This support may have various shapes and configurations including a chair, a zero-gravity chair, an S-shaped chair or bed, and a flat mattress. The wellness system further comprises transducers that are distributed at different locations on or in the chair. These transducers are each capable of receiving a discrete control signal and outputting a single frequency vibration distinct from the frequency vibrations of the other transducers. The transducers typically emit a frequency in the range of 15 hertz to 17,000 hertz, and preferably between 26 hertz and 800 hertz in a frequency that is associated with a match to frequencies of the human body, such as frequencies of the organs, blood stream, bones, etc., as will be understood by persons familiar with sound therapy. The wellness system further comprises at least one speaker capable of emitting music. The wellness system also comprises a control system that sends signals to the transducers and speakers.

### BRIEF DESCRIPTION OF THE DRAWINGS

Preferred embodiments of the invention will now be described by way of example with reference to the accompanying drawings, wherein:

FIG. 1 is a front elevation view of a wellness system including a plurality of transducer spaced in accordance with one embodiment;

FIG. 2 is a front elevation view of the wellness system illustrated in FIG. 1, with portions broken away;

FIG. 3 is a top plane view of the portion of the wellness system illustrated in FIG. 2;

FIG. 4 is a front elevation view of the wellness system illustrated in FIG. 1, with portions broken away;

FIG. 5 is a top plane view of the portion of the wellness system illustrated in FIG. 4;

FIG. 6 is a diagram illustrating one embodiment of a control system of a wellness system; and

FIG. 7 is a diagram illustrating a method of using the control system of the wellness system illustrated in FIG. 6.

### DETAILED DESCRIPTION OF ILLUSTRATIVE EMBODIMENTS

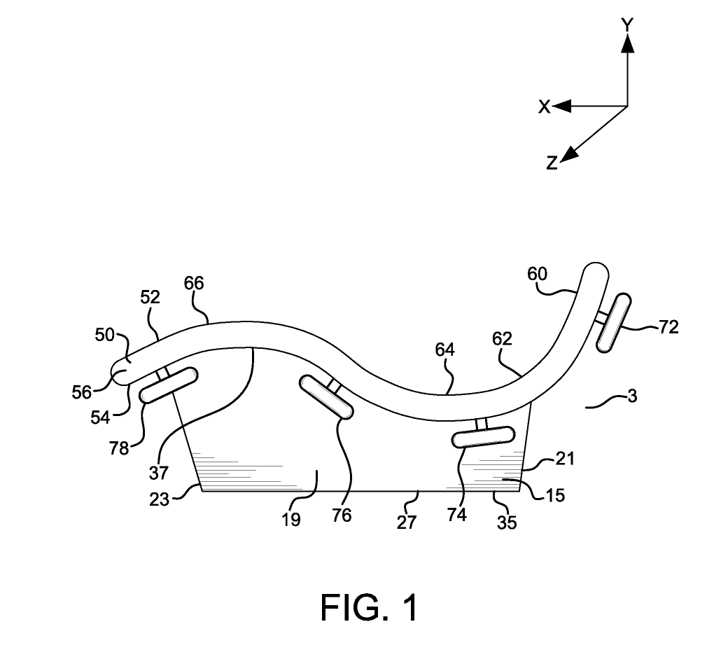

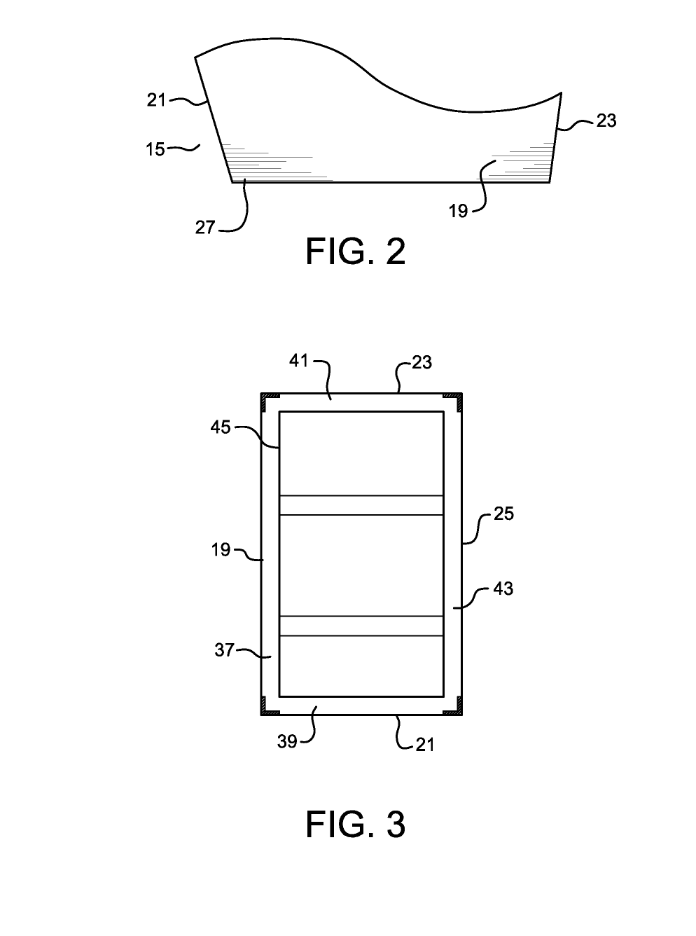

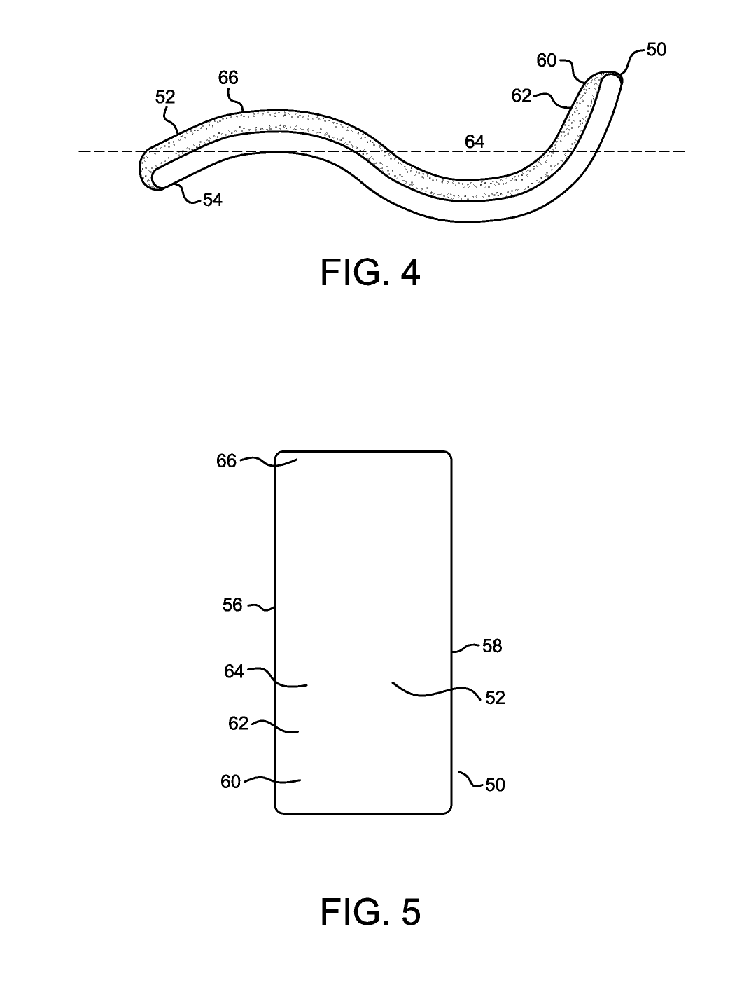

FIGS. 1-5 depict one embodiment of the wellness system 1. FIG. 1 shows a support 3. In this embodiment, support 3 comprises a base 15 and a structure, such as bed 50, for supporting a user. Base 15 has a front 19, a right side 21, a left side 23, and a back 25. Each of front 19, right side 21, left side 23, and back 25 has a bottom edge 27, 29, 31, and 33, respectively. Bottom edges 27, 29, 31, and 33 form base bottom 35. Base bottom 35 sits on a floor or ground (not shown). Each of front 19, right side 21, left side 23, and back 25 also has a top edge 37, 39, 41, and 43, respectively. Top edges 37, 39, 41, and 43 form base top 45.

Support 3 further comprises bed 50. It will be understood by those of skill in the art that the term bed may mean a structure for lying down, sitting, or otherwise supporting at least a portion of a person's body weight. Bed 50 has a top side 52, bottom side 54, front side 56, and back side 58. Bed 50 also comprises a head portion 60, back portion 62, seat portion 64, and foot portion 66. Portions 60, 62, 64, and 66 of support 3 correspond to a person's anatomy in that head portion 60 supports a user's head (not shown), back portion 62 supports a user's back (not shown), seat portion 64 supports a user's seat or buttocks (not shown), and foot portion 66 supports a user's foot or feet (not shown).

It will be understood by those of skill in the art that the configuration of base 15 could be any type of support structure. Base 15 could be comprised of multiple posts of various cross-sectional shape. Base 15 may be larger or smaller relative to the size of bed 50. Additionally, while FIGS. 1-5 show a substantially S-shaped bed as viewed from the front, those of skill in the art will appreciate that bed 50 may be of any shape, curved, slightly curved, or flat as viewed from the front. Bed 50 may be curved, slightly curved or flat as viewed from other perspectives as well. Those of skill in the art will understand that top side 52 may vary in height along the y-axis and z-axis in addition to the x-axis as shown in FIG. 1.

In the present embodiment, wellness system 1 further comprises transducer spaces 72, 74, 76, and 78. Each of the four portions 60, 62, 64, and 66 of bed 50 contain a transducer space. Transducer space 72 is located in head portion 60. Transducer space 74 is located in back portion 62. Transducer space 76 is located in seat portion 64. Transducer space 78 is located in foot portion 66.

The present invention is not limited to the number of location of the transducers shown in FIG. 1 (unless stated in the corresponding claim), as will be understood by persons familiar with wellness systems. Wellness system 1 may also be configured to have transducer spaces that are attached to wellness system 1 by various types of support structures or brackets (not shown).

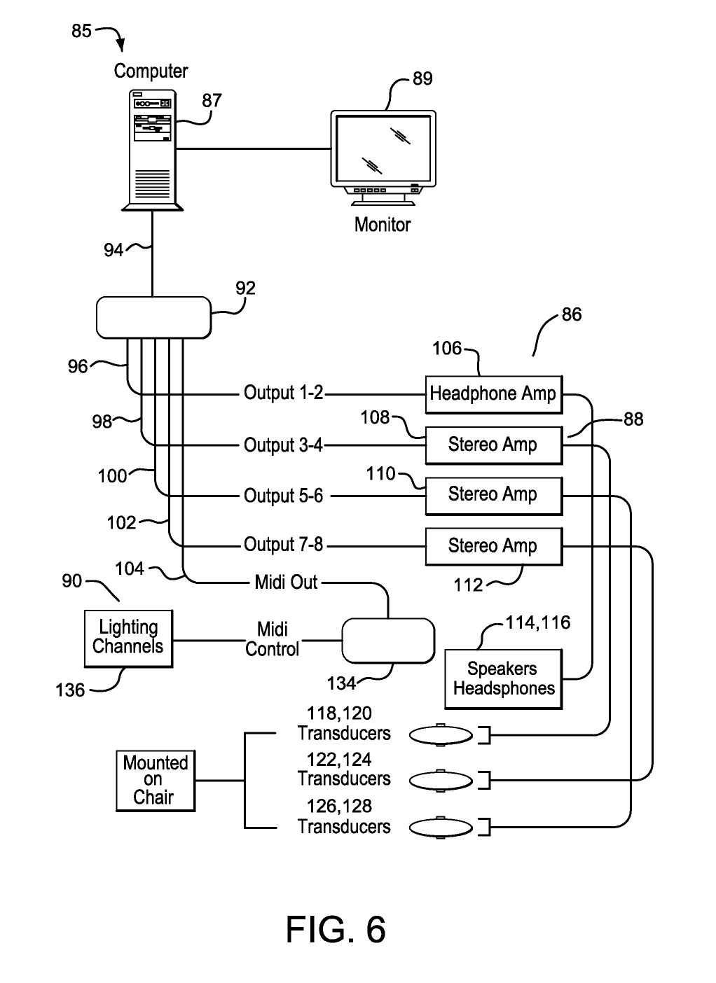

Wellness system 1 further comprises speakers (not shown, but indicated schematically in FIG. 6). Speakers are conventional sound speakers, and preferably are evenly spaced about the user's head. These speakers may be installed in headphones. Alternatively, the speakers may be mounted on or near the support 3. In yet another embodiment, speakers may be placed on a wall of the room containing the wellness system. A person familiar with speaker technology will appreciate that these speakers may be located anywhere on, in, or near the wellness system.

Wellness system 1 further comprises a control system 85. Control system 85 comprises a computer 87. Control system 85 also comprises monitor screen 89. Monitor screen 89 attaches to computer 87.

Control system 85 comprises input/output device 92. Input/output device 92 preferably is the M-Audio ProFire 610 Audio Interface with high-definition 6-in/10-out. Control system 85 may further comprise sound system 86, vibration system 88, and lighting system 90. Input/output device 92 connects to computer 87 by firewall cable 94.

Sound system 86 comprises output connection 96, stereo headphone amplifier 106, and speakers 114 and 116. Output connection 96 connects input/output device 92 to headphone amplifier 106. Headphone amplifier also connects to speakers 114 and 116.

Vibration system 88 comprises output connections 98, 100, and 102, stereo amplifiers 108, 110, and 112, and transducers 118, 120, 122, 124, 126, and 128. Transducers 118, 120, 122, 124, 126, and 128 may be the Clark Synthesis Tactile Sound TST239 Silver Transducers. The frequency response of the TST239 transducer ranges from 15 Hz to 17 kHz. Output connections 98, 100, and 102 connect to input/output device 92. Stereo amplifier 108 connects to output connection 98. Stereo amplifier 110 connects to output connection 100. Stereo amplifier 112 connects to output connection 102. Amplifiers 108, 110, and 112 connect to transducers 118, 120, 122, 124, 126, and 128, respectively. As described above, transducers 118, 120, 122, 124, 126, and 128 and speakers 114 and 116 and may be mounted on support 3.

According to the embodiment shown in FIG. 1, transducer 118 fits in transducer space 72, transducer 120 fits in transducer space 74, transducer 122 fits in transducer space 76, and transducer 124 fits in transducer space 78. Transducers 126 and 128 are optional and may be placed elsewhere on bed 50. Transducers 118, 120, 122, 124, 126, and 128 preferably all operate independently of each other (in both frequency and amplitude). Each are capable of receiving a discrete control signal and outputting a single frequency vibration distinct from the frequency vibrations of the other transducers. The particular frequencies and amplitude output are chosen according to the desired sound therapy treatment. In this way, the vibration of one transducer may be different from or the same as other transducers in wellness system 1.

Additionally, in the present embodiment, lighting system 90 of control system 85 comprises midi 134. Midi 134 connects to input/output device 92 by output connection 104. Midi 134 then connects to lighting channels 136.

Control system 85 further comprises software module 140 that is loaded into the processing unit (not shown) of computer 87 to implement the characteristic features described in detail below. The software module 140 will be described with respect to its functional flow as processed by the processing unit (not shown) of computer 87 and with respect to the flow of information to and from the graphical user interface presented to the user on monitor 89 of control system 85.

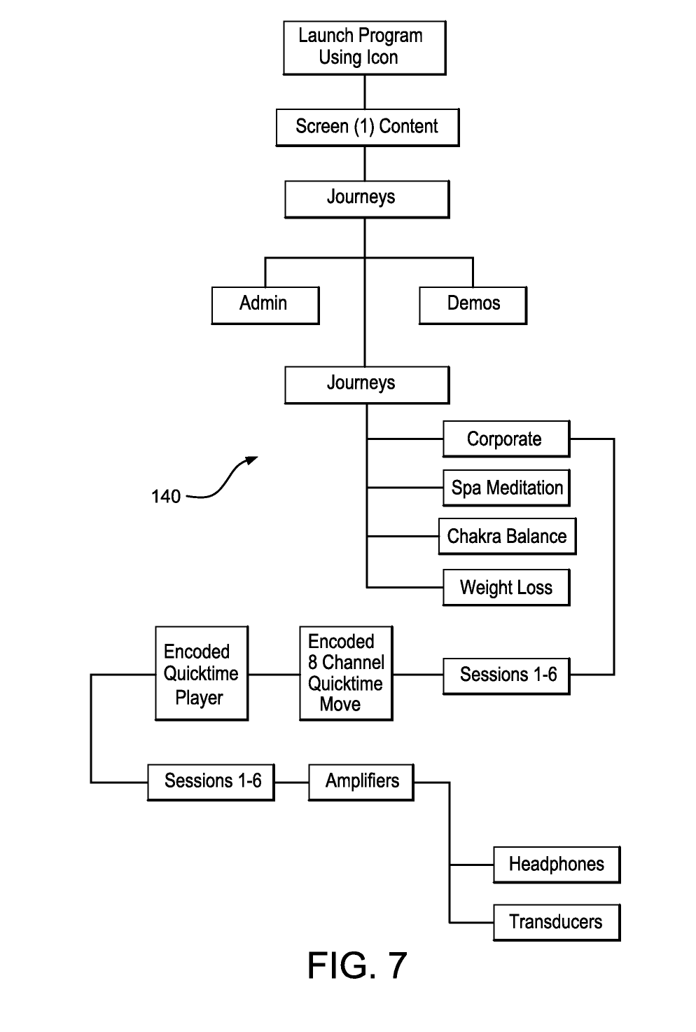

FIG. 7 illustrates the flow of information to and from the graphical user interface. The software is first launched on computer 87. It will be understood by those of skill in the art that computer 87 may be controlled by the user or operator in multiple ways. Monitor 89 may be a touch screen monitor that the user or operator touches to make selections. In another embodiment, computer 87 may be controlled by an external mouse, keyboard, or other similar device. In yet another embodiment, computer 87 may be controlled remotely through an internet or intranet system.

In the illustrated embodiment, once software is launched on computer 87, the software module will display three options: “Admin,” “Journeys,” and “Demo.” The “Journeys” selection will allow the user or operator to choose various types of pre-programmed session types. These may include “Corporate,” “Spa Meditation,” “Chakra Balance,” and “Weight Loss.” The user or operator may further select the number of the pre-programmed session. For example, the “Corporate” session type may list 6 different sessions that a user or operator may select. Once a specific pre-programmed session is selected, this information is encoded and then decoded and sent to the input/output device 92.

In operation, an operator or the user may operate the software that is described above. Control system 85 sends signals to the transducers that correspond to the predetermined frequencies and amplitudes. The single frequency output and amplitude of each transducer are changed and controlled according to the software to enhance wellness of the user according to accepted principles in the sound therapy field.

It should be appreciated that several example embodiments of a wellness system have been described, and that the described examples have been provided for the purpose of explanation and is not to be construed as limiting the invention. For instance, while embodiments have been presented including a control system 85 comprising a lighting system 90, it should be appreciated that the wellness system 1 may or may not include either of these structures.

Although the wellness system 1 has been described with reference to preferred embodiments or preferred methods, it is understood that the words which have been used herein are words of description and illustration, rather than words of limitation. Furthermore, although the wellness system 1 has been described herein with reference to particular structure, methods, and embodiments, the embodiments are not intended to be limited to the particulars disclosed herein, as the invention extends to all structures, methods and uses that are within the scope of the present invention. Those skilled in the relevant art, having the benefit of the teachings of this specification, may effect numerous modifications to the invention as described herein, and changes may be made without departing from the scope and spirit of the invention.

What is claimed:

1. A personal wellness system for supporting a person and subjecting the person to frequencies that are understood by sound therapy practitioners as being associated with synchronization with the body, comprising:
a support for receiving a person comprising a head portion, a back portion, a seat portion, and a foot portion;
plural transducers distributed in the support, each one of the transducers capable of receiving a discrete control signal therefrom and outputting a single frequency vibration;
one or more speakers for outputting music or single frequency sounds;
a menu-driven control system that
sends a signal to the transducers, and
coordinates speaker output with transducer output.

2. The wellness system of claim 1, wherein the single frequency has an amplitude of 26 to 800 hertz.

3. The wellness system of claim 1, wherein the frequencies are synchronized with the left and right brain of the person's body.

4. The wellness system of claim 1, wherein the support is a chair.

5. The wellness system of claim 4, wherein the chair is a zero gravity chair.

6. The wellness system of claim 4, wherein the chair has a substantially S-shaped upper surface.

7. The wellness system of claim 1, wherein the support is a bed.

8. The wellness system of claim 7, wherein the bed has a substantially S-shaped upper surface.

9. The wellness system of claim 7, wherein the bed has a substantially flat upper surface.

10. The wellness system of claim 1, wherein the wellness system comprises four transducers.

11. The wellness system of claim 10, wherein each of the foot portion, seat portion, back portion, and head portion comprise at least one transducer.

12. The wellness system of claim 1, wherein the single frequency ranges from 15 hertz to 1700 hertz.

13. The wellness system of claim 1, wherein the one or more speakers are located near or in the head portion.

14. The wellness system of claim 1, wherein the one or more speakers comprise stand alone speakers.

15. The wellness system of claim 1, wherein the one or more speakers comprise headphones.
